(19) United States
(12) Patent Application Publication
(10) Pub. No.: US 2017/0130193 A1
(43) Pub. Date: May 11, 2017

(54) TISSUE MODEL, METHOD FOR PRODUCING SAID TISSUE MODEL, AND USE OF SAID TISSUE MODEL

(71) Applicant: RWTH AACHEN, Aachen (DE)

(72) Inventors: Andreas BLAESER, Troisdorf (DE); Benjamin THEEK, Aachen (DE); Twan LAMMERS, Maaseik (BE); Fabian KIESSLING, Aachen (DE); Horst FISCHER, Aachen (DE)

(21) Appl. No.: 15/323,119

(22) PCT Filed: Jul. 6, 2015
(86) PCT No.: PCT/EP2015/065319

§ 371 Date: Dec. 30, 2016

(30) Foreign Application Priority Data
Jul. 4, 2014 (DE) 10 2014 109 360.7

(51) Int. Cl. C12N 5/00; C12N 5/09

(52) U.S. Cl. CPC C12N 5/0062; C12N 5/0693; C12N 2503/04; C12N 2503/02; C12N 2533/54; C12N 2533/30

(57) ABSTRACT

In a first aspect, the present application relates to a method for producing a tissue model comprising supply structures. In a further aspect, the application relates to tissue models that can he obtained in this way and to the use of said tissue models as models for tissue genesis, in particular tumorigenesis, including angiogenesis, in the tissue model. Finally, a method for testing and identifying active ingredient candidates, including treatment strategies, is provided.

In a first aspect, the present application is directed to a method for producing a tissue model comprising supply structures. In a further aspect, the application is directed to these thus obtainable tissue models and to the use thereof as models of tissue genesis, especially tumorigenesis, including angiogenesis and the tissue model. Lastly, a method for testing and identifying active-ingredient candidates, including treatment strategies, is provided.

### PRIOR ART

To develop novel active ingredients in, for example, the field of tumor treatment, potential pharmacological substances are investigated via a multistage process, initially in cell culture and, if results are promising, in vivo in animal experiments. A constant finding is that the results of in vitro experiments are, however, not readily transferable to in vivo experiments; many potential active ingredients fail in these experimental phases. A more effective in vitro screening, more particularly an in vitro screening which is successful in relation to the later in vivo experiments, is desirable in order to reduce the effort in developing active ingredients and, at the same time, the numbers of experimental animals. Currently, different 3D in vitro tissue engineering (TE) concepts are being pursued with the goal of establishing an improved screening platform between cell culture and animal experiment. A first step on the way to a 3D tumor model was done with the investigation of statically cultured microparticles loaded with cells, for example Horning J. L., et al. Mol. Pharm. 2008 5: 849-62. With the aid of important key technologies, such as microengineering (e.g., photolithography) and microfluidics, it was possible in the following years to develop more complex hybrid systems composed of synthetic microchannels, membranes and biomaterial-embedded cells; an example that may be mentioned here is Huh, D. et al., 2011, Trends Cell Biol. 21: 745-54. Systems produced in this manner are already being utilized to investigate angiogenesis and tumor cell extravasation, described in, for example, Zheng, Y., et al., Proc. Natl. Acad. Sci USA, 2012; 109: 934-7 or Jeon, J. S. et al. Plos 1 2013; 8: e56910. It has likewise already been possible to develop a microfluidic TE model based solely on biological material: Sekine, H et al. Nat Commun 2013, 4: 1399. In this case, an artery-connected capillary tissue from a donor animal is utilized as a vascular bed for the dynamic culturing of in vitro expanded cells.

Bioprinting is a special tissue engineering method for generating three-dimensional living tissue, which method uses the principle of generative manufacturing processes—also known as rapid prototyping. In contrast to generative manufacturing processes applied in industry, such as stereolithography, selective laser melting or fused deposition modeling for example, bioprinting uses hydrogels loaded with cells as building material. These are applied layer by layer in accordance with a 3D model. Hydrogels mimic the extracellular matrix of natural tissue and thus provide a cell-friendly environment for 3D tissue engineering; see, for example, Lee, K. Y., Chem Ref 2001; 101: 1869-80.

In nonpolymerized form, hydrogels can be easily handled and dispensed. The gel solidifies as a result of a chemical, thermal or light-induced polymerization, making it possible to successively apply subsequent layers. By means of various parameters, such as gel concentration, crosslinker concentration, pH and temperature, it is possible to adjust elasticity and time of polymerization and also rate of polymerization. With the aid of bioprinting, it is possible to construct complex 3D tissue constructs composed of different materials with various cell types, as described in many different ways in the literature, for example Shuurman W., et al., Biofabrication 2011; 3: 021001.

Many methods for forming models of angiogenesis describe techniques in which the various cell types are simultaneously introduced into the matrix.

However, there is a need to improve such three-dimensional tumor angiogenesis models, more particularly to improve the production methods thereof, in order to provide relevant tissue models for screening methods. Here, said models ought to be suitable for investigating tumor growth, tumor angiogenesis and also tumor therapy effects. In this connection, both the tumor or the tissue structure and the supply structure ought to substantially consist of naturally obtained materials, preferably biological material, such as relevant hydrogels and cells. In contrast to the state of the art, it is intended here that individualization of the tissue model be possible.

### BRIEF DESCRIPTION OF THE INVENTION

The present invention provides a method for producing a tissue model comprising supply structures. Said method comprises the steps:
a) applying a composition comprising living cells to or around a preformed first structure in order to form a coating on and optionally complete sheathing thereof around the preformed first structure, the composition being suitable for forming a supply structure as coating;
b) optionally embedding this coated first structure and/or culturing the coated structure in order to form the supply structure;
c) applying a suspension containing living cells to the coating obtained in the previous step or to the supply structure in order to form a tissue structure on the coating or the supply structure and in order to obtain a tissue model;
d) optionally embedding the tissue model obtained in step c).

The present invention further comprises corresponding tissue models and also the use of said tissue models for the analysis of tissue genesis, especially tumorigenesis, including angiogenesis. In addition, the use of the tumor tissue model is suitable for the testing of a possible therapy with a predetermined form of therapy or for the stratification of a selected therapy for the treatment of the tumor, as reflected by the tissue model.

In a further aspect, the application is directed to methods for testing and identifying potential active-ingredient candidates using the tissue model according to the invention in order to identify those potential active-ingredient candidates which exhibit a pharmacological effect on the tissue model.

Said potential active-ingredient candidates are especially those which show, in the tissue model, properties which are cytotoxic, which are cytostatic and which impair supply to the tumor, i.e., which are especially antiangiogenic and anti-inflammatory.

With the aid of a 3D bioprinter, the inventors were successfully able to generate three-dimensional biomimetic tumor angiogenesis models, also referred to hereinafter as 3D-TAM. In this connection, both the supply structure of the tissue model and the tissue structure of the tissue model are produced with the aid of the 3D bioprinting method. The following invention therefore provides, in one embodiment, a tissue structure, such as a tumor, which is artificially constructed and sits on a supply structure, usually a supply vessel, and which can be cultured appropriately and supplied via said supply structure. In this case, angiogenesis is induced in the tissue structure, such as the tumor, and so, starting from the tissue structure, such as the tumor, angiogenesis takes place in order to allow the connection of the tissue structure/the tumor to the supply structure during culturing and to thus improve supply.

This tissue model comprising supply structure allows a reduction in the number of experimental animals, since said model can be utilized after normal cell culture but before first in vivo animal experiments in order, for example, to test potential active-ingredient candidates.

The tissue model obtainable according to the invention is, however, also suitable for developing, with the aid of preliminary investigations carried out in vitro, a tumor therapy strategy individually matched to the patient. Here, the tumor cells for the tissue model and possibly also the cells required for the supply structure are isolated from the individual. Thereafter, the tissue model comprising supply structure is generated with the aid of the method according to the invention in order to test possible forms of therapy on said tissue model and to thus determine the most effective form of therapy for the treatment of the individual.

The biomimetic tissue model according to the invention is distinguished by the fact that both the structure and the composition of the supply structure and the structure and the composition of the tissue structure and thus of the entire tissue model are individually customizable owing to the synthetic production method. This means that, through the application of the 3D bioprinting technology according to the invention, it is possible to individually set the tumor compositions in their nature and diversity of the printed cells and also the structure and composition of the supply structure. Furthermore, as a result of, for example, the individual supply to the tissue model via the supply structure, an extensive and individual testing in relation to the one therapy is possible.

### BRIEF DESCRIPTION OF THE FIGURES

FIG. 1 shows schematically the various steps of the method according to the invention. In FIG. 1A, the first structure is provided on a support. As depicted in FIG. 1B, the composition comprising living cells is then applied according to the invention to said first structure in order to form a supply structure as coating. FIG. 1C shows the inventive tissue model comprising the supply structure and the tissue structure built thereon. FIG. 1D shows a section through the tissue model according to the invention. The inner channel of the supply structure, referred to here as flow channel, allows fluids to pass through. The supply channel has a two-layer structure, an inner layer comprising endothelial cells and an outer layer comprising fibroblasts, in order to be equivalent to a vascular vessel. The tumor tissue of a hydrogel cell suspension comprising tumor cells and fibroblasts has printed structures comprising endothelial cells for investigating the angiogenesis of these structures. FIG. 1E shows the tissue model according to the invention as artificial tumor comprising supply structure, the vessel channel, embedded in an embedding composition. In order for fluids to pass through, the vessel channel is connected to a medium flow using connectors. In this connection, it is, for example, possible to input molecules to be investigated, with the medium, into the tissue model.

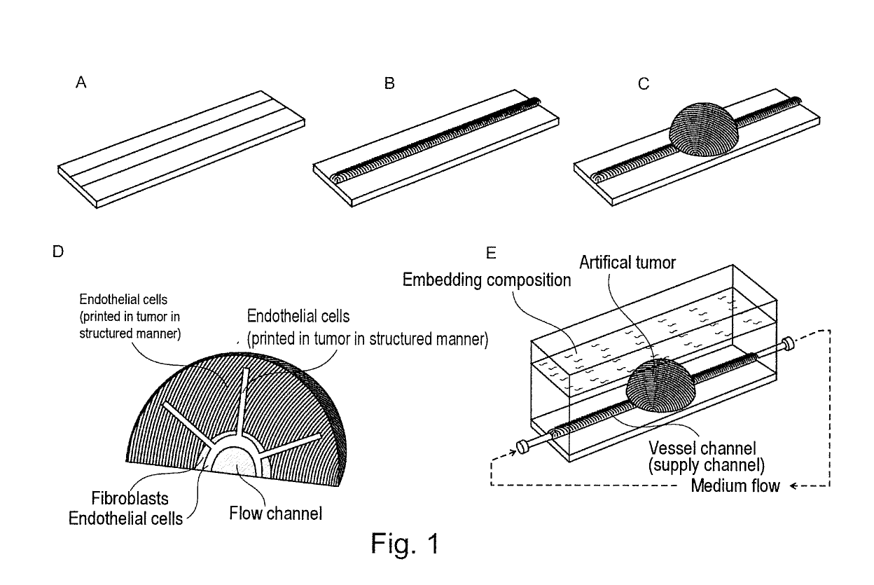

### DETAILED DESCRIPTION OF THE INVENTION

In a first aspect, the present invention is directed to a method for producing a tissue model comprising supply structures, comprising the steps:
a) applying a composition comprising living cells to or around a preformed first structure in order to form a coating on and optionally complete sheathing thereof around the preformed first structure, the composition being suitable for forming a supply structure as coating;
b) optionally embedding this coated first structure and/or culturing the coated structure in order to form the supply structure;
c) applying a suspension containing living cells to the coating obtained in the previous step or to the supply structure in order to form a tissue structure on the coating or the supply structure and in order to obtain a tissue model;
d) optionally embedding the tissue model obtained in step c).

The method according to the invention is distinguished by the fact that the supply structure is produced in a first step, usually by means of a 3D bioprinting method which can be effected by printing, spraying or extrusion. In said first step, a coating is applied to a preformed first structure. Said coating contains living cells for forming the supply structure. Said coating can optionally be a complete sheathing, yielding a supply-vessel-like structure.

The thus obtained, coated first structure can subsequently be embedded in a suitable medium. Suitable media are stated further below. Said coated first structure can be either embedded or further cultured in culture medium in order to form the supply structure, for example in the form of a supply vessel.

The composition comprising living cells for coating the preformed first structure is one which comprises cells and is suitable for forming vessel-forming cells. Said composition contains, in this case, especially endothelial cells, fibroblasts and/or smooth muscle cells or precursor cells thereof.

The expression precursor cells covers multipotent cells and also direct precursor cells of the aforementioned endothelial cells, fibroblasts and/or smooth muscle cells.

The expression “multipotent cells” refers here to those cells which can still differentiate into two or more cell types. Multipotent cells include, for example, totipotent and pluripotent stem cells, induced pluripotent stem cells and embryonic stem cells. Precursor cells include direct precursor cells of the endothelial cells, fibroblasts or smooth muscle cells, including HUVECs (human umbilical vein endothelial cells). In one embodiment, human embryonic stem cells are excluded.

The application to the preformed first structure can be effected in one or more steps or layers. The goal is the formation of the supply structure, such as a vascular structure. In the vascular structures, the internal layer is usually an endothelial cell layer, whereas the outer layer is formed by fibroblasts. Therefore, in one embodiment, a composition comprising endothelial cells or precursor cells thereof is applied to the preformed first structure in a first step and a composition comprising fibroblasts or precursor cells is applied in a further step. Alternatively, these cell types can also be applied in one step.

In one embodiment, the preformed first structure is a removable support material. In another embodiment, said preformed structure can be a solid structure. In again another embodiment, said preformed first structure is shaped on a surface. This is achieved using a removable material, for example biomaterials such as polyethylene glycol, gelatin, fats and waxes or other easily removable materials. Said preformed first structure, also referred to here as channel core, is shaped on a surface by means of a printing method for example. This structure is one which can be removed at a later time, for example by disintegration, flushing, etc. Typically, the first structure is formed in an elongated manner. The first structure can additionally be formed in a branched manner.

In one embodiment, it is further possible, after the removal of the preformed first structure, to further functionalize the supply structure obtained. This includes an introduction of further cells inside and outside the supply structure, but also an inner or outer coating with functional constituents.

Said preformed first structure is then coated with the composition comprising living cells, for example by means of a printing method by printing, spraying or extrusion. In this case, the composition is one which is a liquid/viscous composition at the time of application and which then cures by, for example, polymerization. Materials suitable for this purpose are known hydrogels, including alginate, agarose, hyaluronan, fibrinogen, extracellular matrix components, gelatin, collagen, peptide hydrogels, etc. Here, the materials are natural materials in one embodiment and synthetic materials in another embodiment. In this connection, the gels can be hydrophilic or hydrophobic gels. Suitable hydrogels include those which originate from collagen, hyaluronate, fibrin, alginate, agarose, hydrolysates and combinations thereof. Other suitable hydrogels which are not like the aformentioned ones of natural origin, but are obtained synthetically, include those which originate from polyacrylic acid and derivatives thereof, polyethylene oxides and copolymers thereof, polyvinyl alcohols, polylactic acid, etc. Suitable hydrogels encompass:

Natural hydrogels:

Collagen, fibrinogen, alginate, hyaluronan, gelatin, agarose, chitosan, Matrigel®, hydrogels based on heparin and elastin, including chemically, biologically or physically modified forms of the stated hydrogels.

Synthetic hydrogels:

Polyacrylic acid and the derivatives thereof, polyethylene oxide and the copolymers thereof, polyvinyl alcohol, polyphosphazenes and polypeptides, including chemically, biologically or physically modified forms of the stated hydrogels. In one embodiment, the materials used are biomaterials. In the case of the first structure, which forms a channel core, use is made of a disintegratable or removable material which has in particular noncytotoxic properties or can be removed under noncytotoxic conditions. Gelatin and polyethylene glycols have been found to be suitable for this purpose. In the case of the compositions comprising living cells, hydrogels in particular have been found to be suitable, including alginate, agarose, chitosan, fibrinogen, collagen, etc., such as a combination of agarose and gelatin.

In various specific embodiments, the gel is selected from hydrogel, Novogel™, agarose, alginate, gelatin, Matrigel™, hyaluronan, poloxamer, peptide hydrogels, polyethylene glycol, silicone gels, polylactic acid gels or combinations thereof.

In some embodiments, the hydrogel is a thermoreversible gel, i.e., it is nonliquid at room temperature, but shows a gel-forming temperature at>25 degrees, such as>30 degrees, such as>35 degrees, for example>40 degrees.

The thus present preformed first structure with coating, for example formed in multiple layers, suitable for forming the supply structure is embedded in one embodiment of the invention. As suitable embedding medium, use can be made of possible embedding media as used in 3D bioprinting. This includes in particular compositions consisting of agarose, gelatin, polyacrylamide, polyacrylic acid and the derivatives thereof, polyethylene oxide and the copolymers threof, polyvinyl alcohols, polyphosphazenes, polypeptides, polydimethylsiloxane, polymethyl methacrylate, silicones, and also chemically, biologically or physically modified variants of the stated materials. Suitable compositions include combinations of agarose and gelatin.

In one embodiment, this thus optionally embedded structure is then cultured for a predetermined period in order to allow the formation of the supply structure, for example the formation of vascular vessel structures. Such culturing takes place under known conditions. For example, the duration of such culturing is at least one week, such as a maximum of two weeks for example, for forming the supply structure in order to obtain a vascular vessel similar to a blood vessel.

Said supply structure has an internal channel and is usually formed in an elongated manner. The structure can optionally be formed in a branched manner.

The method according to the invention further comprises the application of a suspension containing living cells to the coating or the postculturing of the supply structure present. After further culturing, the cell suspension forms a tissue structure on the coating or the supply structure in order to thus obtain a tissue model.

In one embodiment, the cells are already present in a suspended manner in the gel to be applied. Alternatively, the gel can first be applied to the coating or to the supply structure formed and the cells are then introduced, for example injected, in a site-specific manner. In one embodiment, these steps can also be combined. This means that further cells, such as tumor cells, are introduced into the tissue structure present that consists of gel and cells.

The expression “tissue model” is understood here to mean: a synthetically produced, cell-containing 3D tissue having at least two substructures, the supply structure and the tissue structure.

The expression “supply structure” is understood here to mean: a synthetically produced, cell-containing 3D cell structure having an inner region which can form a cavity and does not comprise any cells, said inner region having a first inlet and at least one second inlet, it being possible to feed and discharge fluids via said inlets, and an external region containing cells which form a cell structure, such as a cell structure similar to a vascular cell structure. The supply structure is typically formed in an elongated manner. It can optionally be formed in a branched manner.

The expression “tissue structure” is understood here to mean: a synthetically produced 3D cell structure which has cells at least in regions, for example present in the entire structure. The tissue structure is one which comprises, in one embodiment, tumor cells. And also cells, such as precursor cells, which form vessels, such as blood-vessel-type structures, during angiogenesis.

The suspension containing living cells which is applied according to the invention to the coating or the supply structure can be directly applied thereto. In an alternative embodiment, the suspension for forming the tissue structure can be spaced away from the coating or the supply structure. For example, a bioresorbable separating layer can be present between the suspension for forming the tissue structure, and the coating or the supply structure; such a separating layer can, for example, be one based on polylactide, polycaprolactone or other gels, including the gel for embedding the coated first structure. Such spacing may be useful for preventing a premature proliferation of the cells present in the tissue structure or the suspension into the supply structure and for thus possibly avoiding a clogging thereof.

If the supply structure should already be completely formed, for example in the form of a vascular vessel, the suspension containing living cells can be directly applied to said supply structure.

In one embodiment, the living cells present in the suspension comprise tumor cells. To form a tumor tissue model, said tumor cells are applied as tumor tissue structures to the supply structure. Said tumor cells are living tumor cells, for example primary tumor cells isolated from an individual whose therapy is to be stratified or for whom the form of therapy is to be determined on the basis of said tissue model.

Said suspension can further comprise other cell types, especially fibroblasts or endothelial cells, but also smooth muscle cells. These cells are suitable for the formation of new vessels (angiogenesis). The formation of new vessels can take place both within the tissue structure and between tissue structure and supply channel. The latter can ensure a connection of the vessels possibly formed in the tissue structure to the supply channel.

In one embodiment, the tissue structures can be formed such that the entire tissue structure is formed using the same cell suspension. Alternatively, the tissue structure can be formed using more than one suspension of differing composition. As a result, it is possible to form structured tissue structures, for example those in which only tumor cells are present in certain regions or those in which further cell types, such as fibroblasts, smooth muscle cells or endothelial cells, are present in certain regions, in order to portray different tissue structures.

In this connection, the suspension is one which comprises a medium suitable for 3D printing. Said medium is one which is like the abovementioned medium for printing or coating the first structure. Said medium is especially a gel of the abovementioned type. For example, a hydrogel is used, such as collagen, fibrinogen, alginate, hyaluronan, gelatin, agarose, chitosan, Matrigel™, hydrogels based on heparin and elastin, polyacrylic acid and the derivatives thereof, polyethylene oxide and the copolymers thereof, polyvinyl alcohol, polyphosphazenes and polypeptides, including chemically, biologically or physically modified forms of the stated hydrogels. Suitable gels comprise a combination of agarose and gelatin.

In a preferred embodiment, the tissue model is therefore a tumor tissue model having a tissue structure containing tumor cells, and the suspension containing living cells is one which contains tumor cells, such as primary tumor cells.

In one embodiment, the suspension contains living tumor cells and stromal cells, such as endothelial cells, fibroblasts, myofibroblasts, immune cells, including macrophages and leukocytes. The cells are especially primary cells, including stem cells and precursor cells (progenitor cells), of these stated cell types.

In this connection, the suspension is in the form of a hydrogel or of a biocompatible polymer.

The thus obtainable tissue model comprising the tissue structure and the supply structure is subsequently embedded in one embodiment. As embedding media, it is possible to use the abovementioned media, specifically gels, such as agarose, gelatin, polyacrylamide, polyacrylic acid and the derivatives thereof, polyethylene oxide and the copolymers thereof, polyvinyl alcohols, polyphosphazenes, polypeptides, polydimethylsiloxane, polymethyl methacrylate, silicones, and also chemically, biologically or physically modified variants of the stated materials. Alternatively, it is possible to use the tissue model in culture media, as generally known to a person skilled in the art.

In this connection, the embedding composition or the embedding medium, these terms being used here synonymously, is one which is cytocompatible. The expression cytocompatible is understood to mean that the embedding medium or the embedding composition shows a cell and tissue compatibility.

In one embodiment, the application of the suspension containing living cells and/or the application of the composition containing the living cells is done by so-called bioprinting. The expression bioprinting refers to the application of hydrogels loaded with cells. In this case, said hydrogels mimic the extracellular matrix of natural tissue and thus provide a cell-friendly environment for the present production method. In nonpolymerized form, the hydrogels can be accordingly handled easily and solidified by induction, for example chemically, thermally, or by light, by polymerization.

In this connection, the application can be done by means of printing, spraying or extrusion. In one embodiment, the application is done by means of printing, for example by means of a 3D printer.

As noted above, the preformed material forming the first structure can be removed after coating and optionally formation of the supply structure. To utilize the tissue model in which the supply structure serves as supply path for the cells present in the tissue model, said first structure is removed, for example by disintegration thereof. A person skilled in the art is aware of suitable methods for the removal, for example disintegration, of said first structure formed from, for example, gels. Said gels can be gelatin, waxes, fats or readily soluble polymers, such as polyethylene glycol for example. A suitable gel is a combination of agarose and gelatin. By means of appropriate thermal or chemical conditions, said gels can then be disintegrated, forming a channel internally in the supply structure. Through said channel, it is then possible to feed the necessary nutrients, etc., used to supply the tissue model. Furthermore, through the supply structure, it is possible to feed the potential active-ingredient candidates to be tested and also chemical substances, biochemical/biological factors (e.g., cytokines, hormones, growth factors) or further cell types (e.g., macrophages) in order to test the effects thereof on the tissue model.

In this connection, the support to which the first structure is applied or which is integrated in the first structure can be formed such that it is adjustable in temperature, for example can be cooled or can be heated. As a result, it is possible, after coating and curing, to remove the materials forming the first structure. In the case of PEG or gelatin, a heating to 37° C. can lead to a liquefaction thereof, whereas the gel forming the coating, after curing, remains in its form and a channel is thus formed in this supply structure, which allows both the cells in the supply structure and in the tissue structure to be supplied.

In contrast to the prior art, in which the various cell types are introduced simultaneously, the method according to the invention is based on the concept of providing an improved tissue model through sequential formation of the structures.

In a further aspect, the present invention is directed to a thus obtainable tissue model. The tissue model thus obtainable according to the invention is distinguished by the fact that it comprises a supply structure and a tissue structure. A complete supply is possible by means of the supply structure. Both the cells of the supply structure and the cells of the tissue structure are supplied by means of the supply structure. To this end, angiogenesis in the tissue structure/tumor and thus the connection of said tissue structure/tumor to the supply structure is promoted. In this case, different conditions can be simulated depending on the requirements. For instance, hypoxic environments can be provided in an appropriate atmosphere. A long-term culturing of the tissue model can be effected via the supply structure and supply vessels connected thereto. In this case, it is possible, at time-limited time points, to feed additives to the tissue model, for example potential active-ingredient candidates, in order to test the effect thereof on the tissue model. Additionally, various physical or chemical parameters and also cellular parameters can be altered on said tissue model. These include parameters such as pH levels, CO₂ level, pressure, flow rate and temperature, but also the provision of additives, such as growth factors and cytokines and also cells being passed through, etc. The tissue model according to the invention is usable in many different ways. As explained, it is possible to carry out investigations in relation to the influence of potential active-ingredient candidates on the tissue model. In this case, the tissue model can show both tissue structures of healthy origin and tissue structures containing tumor cells or other cells to be treated. The tissue model therefore allows the assessment of an effect of said potential active-ingredient candidates on healthy and altered tissue. It allows a systematic analysis of the various parameters. As a result, it is possible to provide a reproducible model which can, for example, be used in the pharmaceutical industry, for example as an intermediate step between the investigation of potential active-ingredient candidates in cell culture and the subsequent in vivo animal experiments. This makes it possible to substantially reduce the number of animal experiments.

The tissue model according to the invention can be used as a model of tissue genesis, especially tumorigenesis. In particular, it is possible to induce and investigate angiogenesis in tissue.

Another possible use is one in which the tumor tissue model is utilized for the testing in the case of a therapy of a predetermined form of therapy or for the stratification of the therapy for the treatment of a tumor. As a result, it is, for example, possible to develop an individualized form of therapy for an individual to be treated. In this case, the tissue model is established from cells of the individual to be treated, in order to subsequently test in vitro the possible therapies. This has the advantage that the already weakened patient only experiences the form of therapy that is optimal for him, instead of having to be called upon beforehand for the testing of a series of possible therapies, including their adverse effects.

In this connection, it is possible to investigate on the tissue model both antiangiogenic and anti-inflammatory effects and cytostatic and cytotoxic effects. In this case, said tissue model can be constructed according to individual requirements, for example the tissue structure can show various regions having different compositions of cells and cell types. The same applies to the supply structure.

A further substantial advantage of the tissue model according to the invention is the possibility of investigating angiogenesis, tissue genesis and tumor therapy effects with the aid of imaging methods. Said imaging methods include: US (ultrasound), MRI, CT, PET, SPECT (single-photon emission computed tomography), optical imaging methods, including microscopy. Depending on the particular imaging method, it is possible in this case to administer different contrast agents, such as, for example, CT contrast agents, US microbubbles, etc., via the supply structure. The contrast agents can be used for better visualization and contrasting of the 3D-TAM model, of the vessel structure in the 3D-TAM model and also for the detection and localization of specific cell types within the 3D-TAM model. Furthermore, it is, however, also possible to use the 3D-TAM model for developing, investigating and establishing new contrast agents for the abovementioned imaging methods.

Lastly, the present invention provides methods for testing potential active-ingredient candidates. In this case, the method according to the invention comprises the step of providing a tissue model according to the invention and introducing the active-ingredient candidates into the tissue structure via the supply structure and also analyzing the effect of the active-ingredient candidates on the tissue model, with an active-ingredient candidate being identified as potential active ingredient when it exhibits a desired effect on the tissue model.

In one embodiment, said method is one in which the potential active-ingredient candidates are those which exhibit a cytostatic, cytotoxic on tumor cells or those cells which contribute to supplying the tumor, including endothelial cells, fibroblasts and smooth muscle cells, anti-angiogenic and/or anti-inflammatory effect. The method comprises the steps of providing a tissue model according to the invention containing tumor cells, introducing the potential active-ingredient candidates into the tissue structure containing tumor cells via the supply structure, and analyzing the effect of the potential active-ingredient candidates on the tissue model, with a potential active ingredient being identified when it shows an effect which reduces growth of tumors, such as an effect which stops growth of tumors or an effect which makes growth of tumors smaller, an antiangiogenic effect or an anti-inflammatory effect.

### EXAMPLES

The invention will be more particularly elucidated below on the basis of examples without being restricted thereto.

1. Production of a Predetermined First Structure

The preformed first structure is formed on a support surface, for example a culture dish, a glass surface, a base layer identical to the later embedding material (e.g., agarose), or the surface of the bioreactor vessel. This formation can be carried out by printing or extrusion. Here, a gelatin solution is applied using a printer head heated to about 37° C. In a second experiment, polyethylene glycol (PEG) is used as biomaterial.

2. Application of a Coating to a Preformed First Structure

A) A hydrogel-based coating, in this case alginate, mixed with living cells is applied to a first structure composed of gelatin or PEG. To this end, a suspension consisting of alginate (e.g., 2% by weight, sodium salt of alginic acid from brown algae, Sigma) and fibroblasts or HUVECs is applied to the preformed first structure at room temperature with the aid of a 3D printer and subsequently solidified by spraying of a crosslinking solution consisting of calcium chloride (30 mg/ml).

B) A preformed first structure is produced from gelatin or polyethylenge glycol as described above.

With the aid of a 3D printer, a composition composed of alginate (2% by weight, sodium salt of alginic acid from brown algae, Sigma) with endothelial cells is applied to said structure (10 million cells per milliliter) as first layer.

After curing of this alginate layer, a second composition containing fibroblasts (10 million cells per milliliter) is optionally applied as outer layer.

3. Embedding of the Coated First Structure

The thus obtained, coated structure is embedded in agarose (3% by weight).

4. Removal of the First Structure

PEG can be easily removed from the cured coating with the aid of water. The gelatin is liquefied by heating to, for example, 37° C. and sucked off with, for example, the aid of a syringe. During and after removal of the PEG or of the gelatin, the alginate coating loaded with cells is preserved and forms a stable channel wall.

It has been found that the embedded hollow structure coated with alginate and cells prevents a leakage of liquid, and so liquids can be easily passed through the channel thus formed.

A biofunctional tissue which resembles a vascular vessel arises after culturing for approx. two weeks under customary culturing conditions.

5. Introduction of the Tumor Tissue (Completion of the 3D-TAM Model)

The tumor tissue can be introduced into the 3D-TAM model in three different ways.
5.1 Injection of Tumor Cells
The production of the supply channel, the embedding and the culturing are done as described above (points 1-4). After a supply-channel-lining, homogeneous, densely grown endothelial layer has formed, optionally with an additional fibroblast-comprising layer, tumor cells are injected centrally, a few 100 μm above the supply channel, into the embedding composition with the aid of a syringe integrated into the 3D printer.
5.2 Printing of the Tumor onto the Supply Channel
5.2.1 Printing of the Tumor onto a Separating Layer
The supply channel is produced as described (points 1-2). A biodegradable separating layer composed of, for example, polycaprolactone is applied to the supply channel. The tumor tissue in the form of a hydrogel suspension containing tumor cells, for example tumor cells in Matrigel, is printed onto the separating layer. Optionally, the tumor tissue can be structured with further cell types and further hydrogels. The thus produced construct is further processed and cultured as described in points 3-5. Here, the separating layer prevents the proliferation of the tumor cells in the direction of the supply channel during the first weeks of culturing. The separating layer must be designed such that it only disintegrates when the endothelial layer is fully developed in the wall of the channel. After disintegration of the separating layer, access to the supply channel is granted to the tumor tissue and a connection is made possible by means of the formation of new vessels.
5.2.2 Printing of the Tumor after Prior Culturing of the Supply Channel
The production of the supply channel, the embedding and the culturing are done as described (points 1-4). After a supply-channel-lining, homogeneous, densely grown endothelial layer has formed, the construct is removed from the culture and the embedding composition is removed up to a few hundred micrometers above the supply channel (for example, trimming with the aid of a scalpel). The tumor tissue in the form of a hydrogel suspension containing tumor cells, for example tumor cells in Matrigel, is printed onto the surface near the supply channel. Optionally, the tumor tissue can be structured with further cell types and further hydrogels. Thereafter, the entire construct is re-embedded and further cultured.

1. A method for producing a tissue model comprising supply structures, comprising the steps:
a) applying a composition comprising living cells to or around a preformed first structure in order to form a coating on and optionally complete sheathing thereof around the preformed first structure, the composition being suitable for forming a supply structure as coating, the applying step forming a coated first structure;
b) optionally embedding the coated first structure and/or culturing the coated first structure in order to form the supply structure;
c) applying a composition containing living cells to the coating of the coated first structure or to the supply structure in order to form a tissue structure on the coating of the coated first structure or the supply structure and in order to obtain a tissue model with a tissue structure;
d) optionally embedding the tissue model obtained in step c).

2. The method as claimed in claim 1, wherein the supply structure contains or consists of vessel-forming cells.

3. The method as claimed in claim 1, wherein the tissue model is a tumor tissue model having a tissue structure containing tumor cells, and wherein the composition containing living cells contains living tumor cells.

4. The method as claimed in claim 3, wherein the composition containing living cells contains stromal cells.

5. The method as claimed in claim 1, wherein the composition containing living cells is applied in a form of a hydrogel and/or of a biocompatible polymer.

6. The method as claimed in claim 1 wherein the step of applying the composition containing living cells is done by a process selected from the group consisting of means of printing, spraying, and extrusion.

7. The method as claimed in claim 1, wherein the tissue structure in the tissue model comprises different regions or structures.

8. The method as claimed in claim 1, wherein the supply structure and/or the tissue model formed is fixed with a cytocompatible embedding composition.

9. A tissue model obtained using a method as claimed in claim 1.

10. A method of modeling tissue genesis, by providing the tissue model prepared according to claim 9, and monitoring tissue genesis in the tissue model.

11. A method of testing of a therapy with a predetermined form of therapy or for the stratification of the therapy for the treatment of a tumor comprising subjecting the tissue model of claim 9 to the therapy or predetermined form of therapy, and monitoring results of the therapy or predetermined form of the therapy in the tissue model.

12. A method of analysis, comprising comprising subjecting the tissue model of claim 9 imaging methods selected from the group consisting of US, MRI, CT, PET, SPECT, optical imaging, and microscopy.

13. A method for testing and identifying active-ingredient candidates, comprising the step:
a) providing a tissue model as claimed in claim 9;
b) introducing an active-ingredient candidate into the tissue structure via the supply structure and analyzing an effect of the active-ingredient candidate on the tissue model; and
c) identifying the active-ingredient candidate as potential active ingredient when the active-ingredient candidate exhibits a desired effect on the tissue model.

14. The method for testing and identifying active-ingredient candidates as claimed in claim 13, wherein the active-ingredient candidate acts cytostatically, cytotoxically on tumor cells or those cells which contribute to supplying a tumor, and the desired effect is one which reduces growth of tumors, stops growth of tumors, makes tumors smaller, provides an antiangiogenic effect, or provides an anti-inflammatory effect.

15. The method of claim 2 wherein the vessel-forming cells are selected from the group consisting of endothelial cells, fibroblasts, and smooth muscle cells.

16. The method of claim 3 wherein the living tumor cells are primary tumor cells.

17. The method of claim 4 wherein the stromal cells are selected from the group consisting of endothelial cells, fibroblasts, myofibroblasts, immune cells, macrophages, leukocytes, other immune cells, stem cells, progenitor cells, and other primary cells.

18. The method of claim 10 wherein the tissue genesis monitored is tumorigenesis.
